(19) United States
(12) Patent Application Publication
(10) Pub. No.: US 2019/0175482 A1
(43) Pub. Date: Jun. 13, 2019

(54) PROPELLANT-FREE DRY SHAMPOO

(71) Applicant: Henkel AG & Co. KGaA, Dueseldorf (DE)

(72) Inventors: Sandra Brandt, Pinneberg (DE); Marcus Noll, Hamburg (DE)

(73) Assignee: Henkel AG & Co. KGaA, Duesseldorf (DE)

(21) Appl. No.: 16/204,157

(22) Filed: Nov. 29, 2018

(30) Foreign Application Priority Data
Dec. 7, 2017 (DE) 20 2017 222 176.3

(51) Int. Cl. A61K 8/73; A61K 8/89; A61K 8/25; A61K 8/02; A61Q 5/02; A61Q 5/06

(52) U.S. Cl. CPC A61K 8/732; A61K 8/89; A61K 8/25; A61K 8/022; A61K 2800/43; A61Q 5/065; A61K 2800/612; A61K 2800/87; A61Q 5/02

(57) ABSTRACT

The invention relates to a propellant-free dry shampoo, comprising at least one homoglycan product and at least one hydrophobically modified silica. The invention also relates to an atomised powder dispenser comprising a propellant-free dry shampoo, comprising at least one homoglycan product and at least one hydrophobically modified silica. The invention additionally relates to a method for treating keratin fibres and to the use of the propellant-free dry shampoo according to the invention for haircare, for hair washing, in a method for hair dyeing, and/or for temporary reshaping of the hair.

### CROSS-REFERENCE TO RELATED APPLICATION

This application claims priority to German Patent Application No. 20 2017 222 176.3, filed Dec. 7, 2017, which is incorporated herein by reference in its entirety.

### TECHNICAL FIELD

The present disclosure relates to a propellant-free dry shampoo. The present disclosure also relates to an atomised powder dispenser comprising the propellant-free dry shampoo. The present disclosure additionally relates to a method for treating keratin fibres and to the use of the propellant-free dry shampoo for haircare, for hair washing, in a method for hair dyeing, and/or for temporary reshaping of the hair.

### BACKGROUND

Hair cleansing by commercially available shampoos usually forms part of day-to-day bodycare. The purpose of this is to free the hair and the scalp from the skin oils formed in the sebaceous glands, which occur in the form of what is known as sebum from the sebaceous glands. The hair cleansing is also intended to purify the hair and the scalp of the inorganic salts, amino acids, urea and lactic acid secreted from the sweat glands with water, and also of shed skin particles, ambient dirt, odours and possibly residues of cosmetic hair treatments. The conventional shampoos for this purpose contain surfactants and further shampoo ingredients.

As is known in conventional hair cleansing, the hair is moistened or soaked with water, the shampoos is massaged into the hair, and the shampoo is then rinsed out again with water. However, it may be undesirable to have to soak the hair in order to cleanse it, since the hairstyle should not be compromised, as with a wave of water for example. Furthermore, it may be uncomfortable and impossible when away from home to cleanse the hair using a conventional shampoo. Nevertheless, there is a need to cleanse the hair to such an extent that the hair does not have a greasy, unclean appearance.

Dry shampoos offer an alternative to cleansing with conventional shampoos. The objective of the use of dry shampoos is to remove dirt from the hair without destroying the hairstyle by wet washing. Dry shampoos, however, are not shampoos in the conventional sense, since they do not contain typical shampoo ingredients. Their effect is based on the adsorbency and possibly an abrasive effect of powdery particles, which are applied to the dry hair, are massaged into the hair as finely as possible possibly also using a hand towel, and are immediately brushed out once they have soaked up the sebum and the dirt.

Treatment of the hair by surfactant-free, but solvent-containing and propellant-containing dry shampoos that are sprayed onto the hair by employing an aerosol device are known. Starch particles contained in the dry shampoo absorb sebum and dirt once the solvent has evaporated and are brushed out after a reaction time.

The disadvantage of these known dry shampoos lies on the one hand in the use of propellants. For reasons of environmental protection, it is sought to avoid the use of propellants. It is also inconvenient to take propellant-containing spray cans on trips abroad, or this should be avoided due to safety considerations. In order to provide a propellant-free dry shampoo, a dry shampoo composition that can be removed conveniently from a container is required. A powder composition of this kind must be of such a nature that it can be removed from a container for example by a pumping operation, wherein the removal must not be compromised by a clogging of the pump channels as a result of aggregation of the powder composition. It is immediately clear to a person skilled in the art that the removal of powder compositions is technically more difficult to achieve than the removal of a solution or a paste from a container. The type of powder or solid composition itself also plays a key role for a technically advantageous removal of powder compositions, and also for a technically advantageous removal of suspensions having a high solid particle content.

Furthermore, dry shampoos in principle have a lesser cleansing effect and a poorer performance compared to conventional shampoos, and there is thus a need to provide improved dry shampoos. An improvement on the one hand is targeted at an improved cleansing effect, and on the other hand is targeted at enabling an improved shaping of the hair.

The object forming the basis of the present disclosure lies in overcoming the above disadvantages in the prior art, in particular in providing an improved and environmentally friendly dry shampoo which offers greater comfort for the user in respect of its use.

The object forming the basis of the present disclosure is solved by the subject matter of claim 1. A first subject of the present disclosure is therefore a propellant-free dry shampoo, comprising at least one homoglycan product and at least one hydrophobically modified silica.

### BRIEF SUMMARY

In an exemplary embodiment, a propellant-free dry shampoo is provided. The exemplary propellant-free dry shampoo includes at least one homoglycan product and at least one hydrophobically modified silica.

In another embodiment, an atomized powder dispenser is provided. An exemplary atomised powder dispenser includes a propellant-free dry shampoo that includes at least one homoglycan product and at least one hydrophobically modified silica.

In another embodiment, a method is provided for the treatment of keratin fibres. The method includes applying a propellant-free dry shampoo comprising at least one homoglycan product and at least one hydrophobically modified silica to the keratin fibres. Further, the method includes working the keratin fibres using a fabric cloth and/or deforming the keratin fibres.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Within the scope of the present disclosure a dry shampoo shall be understood to mean a composition that is suitable and intended for the cleansing of hair, wherein the dry shampoo is considered a dry shampoo on account of the hair cleansing method, specifically because in the method for cleansing the hair, the hair does not have to be wetted with water or does not have to be completely soaked, in contrast to that which is the case with a conventional shampoo.

The composition comprising at least one homoglycan product and at least one hydrophobically modified silica constitutes a dry shampoo of this kind that can be massaged into the hair without previously soaking the hair. The dry shampoo can be brushed out, but does not have to be, in order to provide cleansed or at least partially cleansed hair. It is even preferred in particular that the dry shampoo is not brushed out and thus contributes to a (temporary) shaping of the hair. Furthermore, the propellant-free dry shampoo is exemplified in that no significant amounts of surfactants are added in an isolated manner, i.e. adsorbed on particles.

Furthermore, merely propellant-free compositions correspond to the present disclosure, i.e. compositions that do not contain any expanding materials by employing which the compositions are driven from a container.

A homoglycan product is a material that constitutes a homoglycan or contains a homoglycan, wherein a homoglycan is a polysaccharide constructed from an equivalent monosaccharide. For example, homoglycans include cellulose or starch. In the propellant-free dry shampoo the homoglycan product is used to absorb pollutants, odorous substances, amino acids, lactic acid and the like and also to adsorb sebum and shed skin particles. The advantage of the use of homoglycans is the availability from natural sources, i.e. they are renewable raw materials.

The propellant-free dry shampoo also contains hydrophobically modified silica. A hydrophobically modified silica within the scope of the present disclosure shall be understood to mean any amorphous or partially amorphous silicon dioxide with silanol groups on the surface thereof that are partially or fully chemically reacted, such that after the reaction the surface is provided at least partially with an organic group. Hydrophobically modified silicas are used in many technical fields and are described in a wide variety in the prior art.

The hydrophobically modified silica has a number of functions in the propellant-free dry shampoo. On the one hand it acts as an abrasive substance, i.e. for removing sebum, dirt particles and shed skin particles. Furthermore, hydrophobically modified silicas contribute to the styling properties of the dry shampoo. In particular, hydrophobically modified silicas have a very good effect on the volume and texture of the hair and also on its suitability for remodelling.

Furthermore, the combination of homoglycan product and hydrophobically modified silica ensures the property that causes the dry shampoo to be able to be removed reliably and comfortably from a dispenser in the form of a propellant-free composition, without tending towards any aggregation. The powder composition as contemplated herein is of such a nature that it can be removed from a container for example by a pumping operation, wherein the removal for example must not be compromised by a clogging of the pump channels as a result of aggregation of the powder particles. The hydrophobically modified silica prevents an aggregation of the solid constituents of the composition and makes the propellant-free dry shampoo pourable, such that it can be removed for example from a pump dispenser without parts of the pump dispenser becoming clogged.

In accordance with a preferred embodiment the homoglycan product is formed from a starch and a surface-active substance. An adsorption product within the scope of the present disclosure shall be understood to mean any product in which a surface-active substance is adsorbed on the surface of the starch. On account of the alcohol groups of the starch, the surface thereof tends to be polar, such that a suspension formed from a starch with a dissolved surface-active substance leads to an adsorption product in which the surface-active substance is present in a form adsorbed physically on the starch particles and thus leads to the adsorption product. Within the scope of the present disclosure a surface-active substance shall be understood to mean any substance that in principle is able to form micelles in water or that, when dissolved in water, reduces the surface tension compared to pure water.

In accordance with a preferred embodiment the starch used for the homoglycan product has an amylose content, based on the total weight of the starch, of less than about 30% by weight. Starch consists usually of amylose, i.e. linear chains with a helical structure that are merely α-1,4-glycosidically linked, and of amylopectin, that is to say heavily branched structures, with α-1,6-glycosidic and α-1,4-glycosidic links. The proportion of amylose compared to amylopectin is heavily dependent on the origin of the starch. There are plant varieties that product starch with a low amylose content compared to others. It has surprisingly been found that the cleansing effect when using starches that have an amylose content of less than about 30% by weight is particularly high. The homoglycan is particularly preferably selected from the group including of corn starch, in particular starch derived from Zea Mays, tapioca starch, and rice starch, in particular rice starch derived from the plant species Oryza Sativa. The homoglycan product particularly preferably contains the homoglycan product rice starch derived from the plant species Oryza Sativa. These starches have proven to be particularly advantageous.

It is not only the amylose content that may play a key role in the origin of the starch. The origin of the starch also has an influence on the size and size distribution of the starch particles and thus on the size of the specific surface of the particles. This also appears to be a reason as to why starch that is obtained from the above-mentioned plat species is particularly well suited for use in the propellant-free dry shampoo.

In accordance with a preferred embodiment of the present disclosure the starch is a dyed starch. Dry shampoos are disadvantageous insofar as the powder leaves behind a haze on the hair. In particular, humans who have dark to black hair thus rule out the use of dry shampoos. The advantage of this preferred embodiment lies in the avoidance of the haze on the hair. The starches used in accordance with this preferred embodiment are produced by mixing dyes with the starch. Since starch is present in the form of particles, dye molecules can be absorbed by the starch particles. The dyes can also deposit on the surfaces of the starch particles.

The homoglycan product is preferably formed from a starch and a surface-active substance. The surface-active substance is preferably a polyorganosiloxane, more preferably a polymethylsilsesquioxane, and/or the surface-active substance is a cationic surfactant, more preferably an alkylammonium salt, even more preferably an alkylammonium salt having at least one linear alkyl group with from about 12 to about 24 C atoms, even more preferably with from about 14 to about 20 C atoms, and in particular a cetyltrimethylammonium salt. Any cosmetically acceptable cation, but preferably the chloride or bromide ion, can be considered to be a counter ion to the alkylammonium cation, in particular to the cetyltrimethylammonium cation. The surface-active substance can also be a dye.

The production of the adsorption product is not critical. For example, it can be produced by dispersing the starch in a medium in which the surface-active substance is present in dissolved form.

The use of an adsorption product has a number of advantages. On the one hand, the adsorption of the surface-active substance changes the polarity of the surface of the homoglycan product. This has an influence on its pourability and its tendency to form aggregates. On the other hand, the surface-active substance changes the ability of the homoglycan product to absorb pollutants and oils from the hair. Polyorganosiloxanes, in particular polymethylsilsesquioxane, have proven to be particularly advantageous for solving the problems forming the basis of the present disclosure. These surface-active substances change the polarity advantageously and make it possible to provide a product that demonstrates a good cleansing performance.

Polyorganosiloxanes are also used in hair-styling products as film formers. It has been surprisingly found that the use of polyorganosiloxanes, in particular of polymethylsilsesquioxane, as surface-active substance on the used starches causes the hair to be easily formable. The preferred embodiment therefore has the advantageous effect of improving the styling results of the hair. This effect can be explained if it is assumed that some of the surface-active substance remains on the surface of the hair.

Alkylammonium salts are therefore advantageous in particular because they hydrophobise the starch particles effectively and at the same time are commercially available particularly readily. This is very particularly true for cetyltrimethylammonium salts.

In accordance with a preferred embodiment the homoglycan product is (a) a first adsorption product of starch, in particular rice starch, with the alkylammonium salt, in particular with the cetyltrimethylammonium salt, (b) a second adsorption product of starch, in particular tapioca starch, with the polyorganosiloxane, in particular polymethylsilsesquioxane, or a mixture of (a) and (b), more preferably a mixture of (a) and (b) in a weight ratio of (a) to (b) of from about 40:1 to about 5:1, more preferably of from about 30:1 to about 10:1.

This preferred embodiment and in particular the use of two different adsorption products as homoglycan products enables a tailored adaptation of the desired properties. An adsorption product formed from rice starch with an alkylammonium salt has proven to be advantageous to adjust the hydrophobicity of the particles and thus the pourability of the produced propellant-free dry shampoo. An adsorption product formed from tapioca starch with a polyorganosiloxane is advantageous for the hair-styling properties of the hair treated with the propellant-free dry shampoo. The use of a mixture of these adsorption products is thus advantageous for solving the problems forming the basis of the present disclosure. In particular with the more preferred mixture, advantageous properties can thus be controlled in a targeted manner.

In a preferred propellant-free dry shampoo the hydrophobically modified silica is a styling product formed from a silica, in particular a pyrogenic silica, and an organo-silicon compound. Silylation reactions of silicas with organo-silicon compounds are known and are used industrially on a large scale. Here, the organo-silicon compounds are covalently bonded to the silicas by reacting the silanol groups of the silicas with the organo-silicon compounds. In accordance with a preferred embodiment the organo-silicon compound is an alkylsiloxane or a silazane, wherein the organo-silicon compound is preferably an alkylsiloxane, in particular hexamethyldisilazane. The materials are the best suitable for the reaction. Hydrophobisation with hexamethyldisilazane is the most conventional method, and the hydrophobised products are the most widespread available on a commercial scale.

Examples are Aerosil® R104 V, Aerosil® R106, Aerosil® R202, Aerosil® R805, Aerosil® R812, Aerosil® R812S, Aerosil® R972 and Aerosil® R8200, all from Degussa, and HDK® H2000, HDK® H2050 and HDK® H3004, all from Wacker. To summarise, hydrophobically modified silicas that are selected from the compounds with the INCI names Silica Dimethyl Silylate (for example Aerosil® R792, Aerosil® R794), Silica Dimethicone Silylate (for example Aerosil® R202), Silica Caprylyl Silylate (for example Aerosil® R805) and Silica Silylate (for example Aerosil® R812 or Aerosil® R816) are preferred. Compounds with the INCI name Silica Silylate are particularly preferably used.

Pyrogenic silicas are particularly readily available on a commercial scale. Their production is known and described in detail in the prior art.

In accordance with a preferred embodiment the homoglycan product is contained, based on the total weight of the propellant-free dry shampoo, in an amount of from about 15 to about 60% by weight, preferably from about 20 to about 50% by weight, more preferably from about 25 to about 35% by weight, in the propellant-free dry shampoo.

These amounts have proven to be particularly advantageous for solving the problem forming the basis of the present disclosure.

In accordance with a preferred embodiment the hydrophobically modified silica is contained, based on the total weight of the propellant-free dry shampoo, in an amount of from about 4 to about 40% by weight, preferably from about 8 to about 25% by weight, more preferably from about 10 to about 20% by weight, in the propellant-free dry shampoo.

These amounts have proven to be particularly advantageous for solving the problem forming the basis of the present disclosure. In the preferred embodiments that are exemplified by the specified amounts of homoglycan product and hydrophobically modified silica, it must be taken into consideration that the sum thereof is not 100 in every case. In such embodiments further ingredients or liquid media are contained in the propellant-free dry shampoo.

In a particular embodiment of the present disclosure the propellant-free dry shampoo also contains a dye and/or pigments, in particular surface-modified pigments. This embodiment offers advantages in the case of permanently dyed hair. As explained above, dry shampoos are associated with disadvantages since the powder can leave behind a haze on the hair. A dye or a pigment can prevent this haze.

In accordance with a preferred embodiment of the present disclosure the propellant-free dry shampoo also contains a film former, preferably an acrylate-based polymer as film former, more preferably a copolymer at least formed from the monomer units octylacrylamide, acrylic acid and butylaminoethyl methacrylate as film former. A film former is understood to mean polymers that can leave behind a continuous film on the skin, the hair or the nails as it dries. Film formers of this kind can be used in a wide range of different cosmetic products, such as face masks, make-up, hair fixers, hairsprays, hair gels, hair waxes, hair conditioners, shampoos or nail varnishes. Within the scope of the present disclosure a film former is understood to mean in particular polymers that, when applied in from about 0.05 to about 29% by weight (based on the total weight of the cosmetic agent) aqueous, alcoholic or aqueous-alcoholic solution, are able to despot on the hair in the form of a transparent polymer film. Within the scope of the present disclosure, acrylate-based film formers are preferred. These include all polymers that are constructed from acrylate or methacrylate monomer units. A copolymer that is constructed at least from the monomer units octylacrylamide, acrylic acid and butylaminoethyl methacrylate is preferred. This copolymer is a terpolymer. A copolymer that is constructed from these three monomers and one further monomer can also be used. An example is the copolymer Amphomer® from AkzoNobel Surface Chemistry).

The film former is contained as powder in the propellant-free dry shampoo. By incorporating the propellant-free dry shampoo into the hair, one of the film formers remains on the hair and thus ensures an improvement of the hair styling, in particular an improved hold of the hairstyle.

In a further preferred embodiment of the present disclosure the propellant-free dry shampoo, based on the total weight of the propellant-free dry shampoo, has a water content of about 70% by weight and less, preferably of about 60% by weight and less, more preferably of less than about 50% by weight. The propellant-free dry shampoo may also comprise an aqueous medium, for example an alcoholic-aqueous medium.

The fact that the shampoo is a dry shampoo does not necessarily mean that purely powder compositions are concerned. Here, reference is made to the above comments regarding dry shampoos. The use of water or aqueous solvents enables improved workability into the hair. The ingredients can be better distributed in the hair, and the ability to style the hair is improved. The removal or partial removal of the propellant-free dry shampoo—if desired—is also significantly improved.

A second subject of the present disclosure is an atomised powder dispenser comprising a propellant-free dry shampoo as contemplated herein.

Atomised powder dispensers are known in the prior art.

The propellant-free dry shampoos can be dosed for their application using an atomised powder dispenser of this kind. The atomised powder dispensers as contemplated herein offer improved possibilities for providing the hair with a fresher look, even without the usual full washing, and/or for temporarily deforming it.

In accordance with a preferred embodiment of the present disclosure the propellant-free dry shampoo is present in the form of powder in the atomised powder dispenser. The advantage of this preferred embodiment is substantiated in the fact that the active ingredient composition, that is to say the propellant-free dry shampoo, is a solid and therefore the hair does not have to be wetted for a cleansing procedure.

A third subject of the present disclosure is a method for treating keratin fibres, in particular human hair, comprising the following steps:
applying the propellant-free dry shampoo as contemplated herein to the keratin fibres, in particular to the human hair, in a first step; and
working the keratin fibres, in particular human hair, with a fabric cloth and/or shaping the keratin fibres in a second step.

As a result of the method, hair can be cleansed and optionally temporarily shaped using the propellant-free dry shampoo. In the first step the propellant-free dry shampoo is applied to the hair. The propellant-free dry shampoo is preferably mixed in intensely using the hands and is left to take effect for a certain period of time. In a second step the hair can be worked using a fabric cloth. The term “work” is understood preferably to mean a mechanical working or treatment. The propellant-free dry shampoo is thus distributed uniformly in the hair. The fabric cloth can be a conventional hand towel, a disposable towel, or the like.

Alternatively, the propellant-free dry shampoo is distributed in the hair without prior treatment with a fabric cloth, and the hair is deformed or reshaped in the second step into a desired hairstyle. Preferred embodiments of the present disclosure comprise a homoglycan product that comprises a polyorganosiloxane. This homoglycan product makes it possible for the hair to be shaped.

The method is in particular a method for dry cleansing and/or temporary deforming of keratin fibres, in particular human hair.

A fourth subject of the present disclosure is the use of a propellant-free dry shampoo as contemplated herein for haircare, for hair washing, in a method for hair dyeing, and/or for temporarily reshaping hair.

Cosmetic agents that are very particularly preferred as contemplated herein comprise at least one of the following embodiments A) to L):

### A)

A propellant-free dry shampoo, comprising at least one homoglycan product and at least one hydrophobically modified silica, wherein the homoglycan product is an adsorption product formed from a starch and a surface-active substance.

### B)

A propellant-free dry shampoo, comprising at least one homoglycan product and at least one hydrophobically modified silica, wherein the homoglycan product comprises a starch selected from the group including of corn starch, in particular starch derived from Zea Mays, tapioca starch, and rice starch, particularly preferably is rice starch derived from the plant species Oryza Sativa.

### C)

A propellant-free dry shampoo, comprising at least one homoglycan product and at least one hydrophobically modified silica, wherein the homoglycan product is an adsorption product formed from a starch and a surface-active substance, wherein the starch is selected from the group including of corn starch, in particular starch derived from Zea Mays, tapioca starch, and rice starch, particularly preferably is rice starch derived from the plant species Oryza Sativa.

### D)

A propellant-free dry shampoo, comprising at least one homoglycan product and at least one hydrophobically modified silica, wherein the homoglycan product is an adsorption product formed from a starch and a surface-active substance, wherein the surface-active substance is a polyorganosiloxane, or wherein the surface-active substance is a cationic surfactant, in particular a cetyltrimethylammonium salt.

### E)

A propellant-free dry shampoo, comprising at least one homoglycan product and at least one hydrophobically modified silica, wherein the homoglycan product (a) is a first adsorption product of rice starch with the alkylammonium salt and (b) a second adsorption product of tapioca starch with the polyorganosiloxane, wherein the mixture of (a) and (b) is present in the weight ratio (a) to (b) of from about 40:1 to about 5:1, more preferably of from about 30:1 to about 10:1.

### F)

A propellant-free dry shampoo, comprising at least one homoglycan product and at least one hydrophobically modified silica, wherein the homoglycan product is an adsorption product formed from a starch and a surface-active substance, wherein the hydrophobically modified silica is a silylation product formed from a silica, in particular a pyrogenic silica, and an organo-silicon compound.

### G)

An atomised powder dispenser, containing a propellant-free dry shampoo, comprising at least one homoglycan product and at least one hydrophobically modified silica, wherein the homoglycan product is an adsorption product formed from a starch and a surface-active substance, wherein the hydrophobically modified silica is a silylation product formed from a silica, in particular a pyrogenic silica, and an organo-silicon compound.

### H)

An atomised powder dispenser, containing a propellant-free dry shampoo, comprising at least one homoglycan product and at least one hydrophobically modified silica, wherein the homoglycan product is an adsorption product formed from a starch and a surface-active substance, and wherein the hydrophobically modified silica is a silylation product formed from a silica, in particular a pyrogenic silica, and an organo-silicon compound, wherein the propellant-free dry shampoo has a water content of about 70% by weight and less, preferably of about 60% by weight and less, more preferably of less than about 50% by weight.

### I)

A propellant-free dry shampoo, comprising at least one homoglycan product and at least one hydrophobically modified silica, wherein the homoglycan product comprises a dyed starch selected from the group including of dyed tapioca starch and dyed rice starch, particularly preferably dyed rice starch derived from the plant species Oryza Sativa.

### J)

A propellant-free dry shampoo, comprising at least one homoglycan product, at least one hydrophobically modified silica, and at least one film former, wherein the homoglycan product comprises a starch selected from the group including of tapioca starch and rice starch, particularly preferably is rice starch derived from the plant species Oryza Sativa.

### K)

A propellant-free dry shampoo, comprising at least one homoglycan product, at least one hydrophobically modified silica, and at least one film former, wherein the homoglycan product comprises a starch selected from the group including of tapioca starch and rice starch, particularly preferably is rice starch derived from the plant species Oryza Sativa, wherein the film former is an acrylate-based polymer.

### L)

A propellant-free dry shampoo, comprising at least one homoglycan product, at least one hydrophobically modified silica, and at least one film former, wherein the homoglycan product comprises a starch selected from the group including of tapioca starch and rice starch, particularly preferably is rice starch derived from the plant species Oryza Sativa, wherein the film former is a copolymer formed at least from the monomer units octylacrylamide, acrylic acid and butylaminoethyl methacrylate.

Features relating to preferred embodiments of the first subject of the present disclosure that have been described above only in that respect of course apply similarly as features of preferred embodiments for the second to fourth subjects.

The following example is intended to explain the subject matter of the present disclosure without in any way limiting it.

### EXAMPLE

A composition as contemplated herein was produced in accordance with the following formulation:

| Raw material | E1* |
|---|---|
| Water | 52 |
| Aerosil R 812 S¹⁾ | 14 |
| Rice starch D.S.A. 7²⁾ | 32 |
| Dry-Flo TS Pure³⁾ | 2 |

*values in % by weight Active substance
¹⁾Silica Silylate (INCI)
²⁾Oryza Sativa rice starch, Cetrimonium Chloride (INCI)
³⁾tapioca starch, polymethylsilsesquioxane

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A propellant-free dry shampoo, comprising
at least one homoglycan product and
at least one hydrophobically modified silica.

2. The propellant-free dry shampoo according to claim 1, wherein the homoglycan product is an adsorption product formed from a starch and a surface-active substance.

3. The propellant-free dry shampoo according to claim 1, wherein the starch has an amylose content, based on the total weight of the starch, of less than about 30% by weight.

4. The propellant-free dry shampoo according to claim 2, wherein the starch is selected from the group consisting of corn starch, tapioca starch, and rice starch.

5. The propellant-free dry shampoo according to claim 2, wherein the starch is a dyed starch.

6. The propellant-free dry shampoo according to claim 2, wherein the surface-active substance is a polyorganosiloxane and/or wherein the surface-active substance is a cationic surfactant.

7. The propellant-free dry shampoo according to claim 1, wherein the homoglycan product is (a) a first adsorption product of starch with the alkylammonium salt, (b) a second adsorption product of starch with the polyorganosiloxane, or a mixture of (a) and (b).

8. The propellant-free dry shampoo according to claim 1, wherein the hydrophobically modified silica is a silylation product formed from a silica, and an organo-silicon compound, wherein the organo-silicon compound is an alkylsiloxane or a silazane.

9. The propellant-free dry shampoo according to claim 1, wherein the homoglycan product, based on the total weight of the propellant-free dry shampoo, is included in the propellant-free dry shampoo in an amount of from about 15 to about 60% by weight and/or wherein the hydrophobically modified silica, based on the total weight of the propellant-free dry shampoo, is included in the propellant-free dry shampoo in an amount of from about 2 to about 30% by weight.

10. The propellant-free dry shampoo according to claim 1, wherein the propellant-free dry shampoo further comprises a dye and/or a pigment; and/or wherein the propellant-free dry shampoo further comprises a film former.

11. The propellant-free dry shampoo according to claim 1, wherein the propellant-free shampoo, based on the total weight of the propellant-free dry shampoo, has a water content of no more than about 70% by weight.

12. An atomised powder dispenser, comprising a propellant-free dry shampoo, wherein the propellant-free dry shampoo comprises at least one homoglycan product and at least one hydrophobically modified silica.

13. The atomised powder dispenser of claim 12, wherein the propellant-free dry shampoo is present in powder form.

14. A method for the treatment of keratin fibres, the method comprising:
applying a propellant-free dry shampoo comprising at least one homoglycan product and at least one hydrophobically modified silica to the keratin fibres; and
working the keratin fibres using a fabric cloth and/or deforming the keratin fibres.

15. The method of claim 14, wherein the treatment of the keratin fibers includes hair washing.

16. The method of claim 14, wherein the treatment of the keratin fibers includes hair dyeing.

17. The method of claim 14, wherein the treatment of the keratin fibers includes temporary reshaping of the keratin fibers.
